(19) United States
(12) Patent Application Publication
(10) Pub. No.: US 2006/0111647 A1
(43) Pub. Date: May 25, 2006

(54) Urine specimen container for female patients

(76) Inventors: Julie Starling, Savannah, GA (US); Sara Phillips, Jekyll Island, GA (US)

Correspondence Address: SMITH, GAMBRELL & RUSSELL, LLP, 1230 PEACHTREE STREET, N.E., SUITE 3100, PROMENADE II, ATLANTA, GA, 30309-3592, US

(21) Appl. No.: 10/992,952

(22) Filed: Nov. 19, 2004

(51) Int. Cl. A61B 5/00

(52) U.S. Cl. 600/574

(57) ABSTRACT

The female urine specimen container of the present invention comprises a urine collection cup having an opening contoured to fit the female anatomy having a generally triangular shape with rounded corners, a bottom having a generally triangular shape with rounded corners, a front wall and a pair of side walls extending downwardly from the opening to the bottom, and a backwardly protruding portion formed by the junction of the pair of side walls. The backwardly protruding portion corresponds to the narrowest portion of the opening and the front wall corresponds to the broadest portion of the opening.

### FIELD OF THE INVENTION

The present invention is directed to containers for collecting specimens from patients. More specifically, the present invention is directed to a urine specimen container for female patients, especially pregnant patients.

### BACKGROUND OF THE INVENTION

Urinalysis is an important and routine procedure for diagnosis and monitoring of various medical conditions as well as for drug testing. The common practice for collection of a specimen is to provide the patient with a paper or plastic cup to be held by hand in the urine stream. While this tends to cause little difficulty for males, for most females, and particularly pregnant women, there are significant difficulties associated with using a standard cup for collecting the sample.

In one instance, the shape of the cup does not anatomically match the female genitalia and, in addition, must be held away from body to prevent extraurethral contamination. This results in some or all of a sample failing to be collected in the cup because positioning of the cup is most often achieved by trial and error. This results in a messy collection process that also tends to be very unsanitary as the patient quite often accidentally urinates on her hands and on the outside of the collection cup.

In another instance, due to the lack of a handle, and especially with pregnant patients, it is awkward to hold the cup, again leading to unclean collection processes that are not sanitary.

Accordingly, the present invention is designed to satisfy one or more of the previous problems. The present invention provides a urine specimen container that is beneficially useful for all female patients, and especially pregnant patients. The urine specimen container improves patient comfort, makes the collection process easier, and makes the collection process more sanitary.

### SUMMARY OF THE INVENTION

It is an advantage of the present invention to provide a urine specimen container that is gender appropriate for females.

It is another advantage of the present invention to provide a urine specimen collection device with increased ease of use for the collection process and cleaner specimen collection for patients, especially pregnant patients.

In the exemplary embodiment, the female urine specimen container comprises a urine collection cup having an opening contoured to fit the female anatomy having a generally triangular shape with rounded corners, a bottom having a generally triangular shape with rounded corners, a front wall and a pair of side walls extending downwardly from the opening to the bottom, and a backwardly protruding portion formed by the junction of the pair of side walls. The backwardly protruding portion corresponds to the narrowest portion of the opening and the front wall corresponds to the broadest portion of the opening. The opening preferably has a lip that is rounded to provide a smooth surface. In other embodiments, the lip is flared from the top of the front wall and side walls for directing the sample into the cup during the collection process. The cup has a handle extending from the front wall to assist the patient in holding the cup and to increase the likelihood of a clean collection process, thereby decreasing the likelihood that the collection process will be unsanitary.

In another embodiment, the inventive female urine specimen container includes a backsplash shield formed by extending the bottom portion of the handle upward at the backwardly protruding portion of the cup. The backsplash shield further increases the likelihood of a clean and sanitary collection process.

In yet another embodiment, the female urine specimen container of the present invention includes a handle that is connected to the front wall through a brim for providing further support and balance when holding the cup.

These and further advantages of the present invention will become apparent after a review of the following detailed description of the disclosed embodiments.

### BRIEF DESCRIPTION OF THE DRAWINGS

FIG. 1 is a top plan view of a urine specimen container according to a first embodiment of the present invention.

FIG. 2 is a left side elevation view of a urine specimen container according to a first embodiment of the present invention.

FIG. 3 is a top plan view of a urine specimen container according to a second embodiment of the present invention.

FIG. 4 is a left side sectional view of a urine specimen container according to a second embodiment of the present invention.

FIG. 5 is a perspective view of a urine specimen container according to a second embodiment of the present invention.

### DETAILED DESCRIPTION OF THE INVENTION

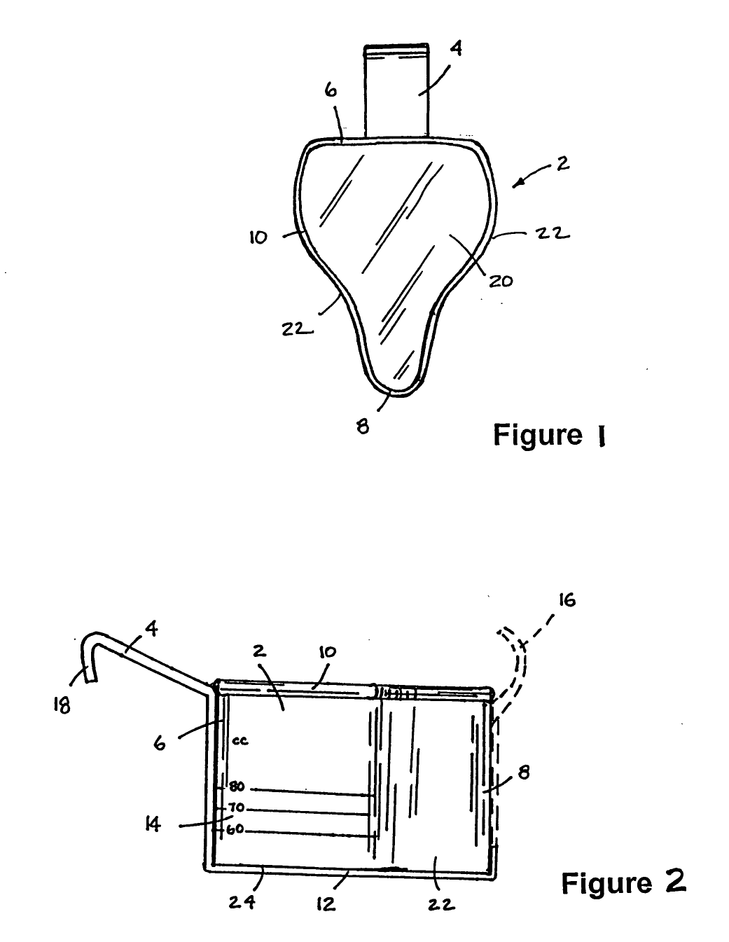

As illustrated in FIGS. 1 and 2, the female urine specimen container comprises a urine collection cup 2 having an opening 20 contoured to fit the female anatomy having a generally triangular shape with rounded corners, a bottom 24 having a generally triangular shape with rounded corners, a front wall 6 and a pair of side walls 22 extending downwardly from the opening 20 to the bottom 24, and a backwardly protruding portion 8 formed by the junction of the pair of side walls 22. The backwardly protruding portion 8 corresponds to the narrowest portion of the opening 20 and the front wall 6 corresponds to the broadest portion of the opening 20 to ensure the catching of urine streams that may be slightly angled.

The opening 20 preferably has a lip 10 that is rounded to provide a smooth surface. The urine specimen container preferably includes a handle 4 extending from the front wall 6 of the cup 2 to assist the patient in holding the cup 2 and to increase the likelihood of a clean and sanitary collection process. The cup 2 may be formed from plastic, polyethylene, or other appropriate material. Volume indicators 14 may be printed or embossed on the pair of side walls 22.

The handle 4 is shaped to facilitate gripping and supporting the cup 2 and may include a loop or a hook 18 as shown in FIG. 2. In one embodiment, the handle 4 has a bottom portion 12 which essentially wraps around and supports the bottom 24 of the cup 2. The handle 4 can be formed integrally with the cup 2 or, it can be formed as a separate component so that the cup 2 can be removed and the handle 4 re-used with a new cup 2 after the handle 4 has been sterilized. With the cup 2 and handle 4 formed as separate units, the cost of manufacture of the specimen container can be reduced by simplifying the molds. The cup 2 can be made from a disposable material, such as lightweight plastic or plastic-coated paper, to reduce product cost and prevent possible contamination of a sample from improperly cleaned containers.

In an alternate embodiment illustrated by the dashed lines in FIG. 2, a backsplash shield 16 may be formed by extending the bottom portion 12 of the handle 4 upward at the backwardly protruding portion 8 of the cup 2. The shield 16 is particularly helpful in preventing spillage of the sample as the container is lifted immediately after collection of the sample.

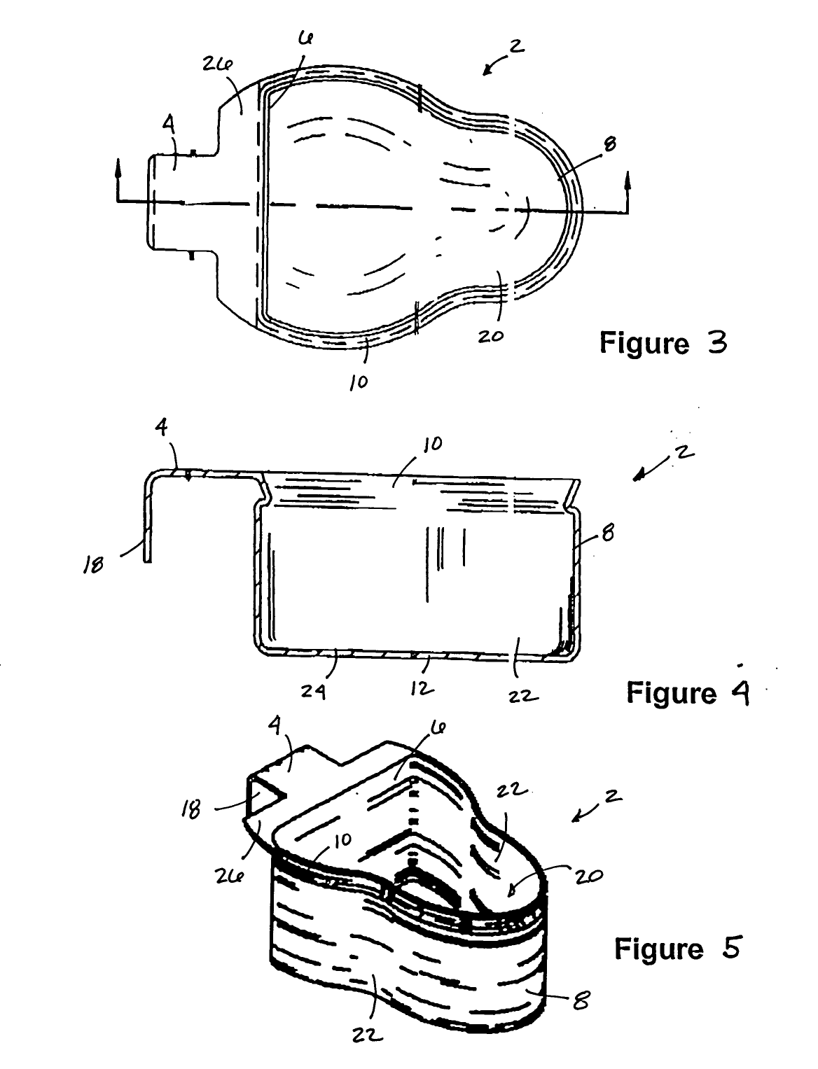

With reference to FIGS. 3-5, in another embodiment of the present invention, the handle 4 is formed integrally with the cup 2 and is connected to the front wall 6 through a brim 26. The brim 26 provides further support and balance for holding the cup 2. Further, in the described embodiment, the lip 10 of the opening 20 is flared from the top of the front wall 6 and side walls 22. Such flaring of the lip 10 aids in directing the sample into the cup 2 during the collection process.

Having thus described the invention in detail, it should be apparent that various modifications and changes may be made without departing from the spirit and scope of the present invention. Consequently, these and other modifications are contemplated to be within the spirit and scope of the following claims.

I claim:

1. A urine specimen container for a female comprising:
a. a urine collection cup having
i. an opening contoured to fit the female anatomy having a generally triangular shape with rounded corners,
ii. a bottom having a generally triangular shape with rounded corners,
iii. a front wall extending downwardly from said opening to said bottom,
iv. a pair of side walls extending downwardly from said opening to said bottom, and
v. a backwardly protruding portion being formed by the junction of said pair of side walls;
wherein said backwardly protruding portion corresponds to the narrowest portion of said opening; and
wherein said front wall corresponds to the broadest portion of said opening.

2. The urine specimen container of claim 1, wherein the urine specimen container further includes a handle.

3. The urine specimen container of claim 2, wherein the handle is formed integrally with the urine collection cup.

4. The urine specimen container of claim 2, wherein the handle and the urine collection cup are formed separably.

5. The urine specimen container of claim 2, wherein the urine specimen container further includes a backsplash shield.

6. The urine specimen container of claim 2, wherein the handle is connected to the front wall through a brim.

7. The urine specimen container of claim 1, wherein the opening includes a rounded lip.

8. The urine specimen container of claim 1, wherein the opening includes a flared lip.

9. The urine specimen container of claim 1, wherein the urine specimen container comprises a plastic material.
